(19) United States
(12) Patent Application Publication
(10) Pub. No.: US 2013/0149270 A1
(43) Pub. Date: Jun. 13, 2013

(54) ALKALINE HAIR RELAXING COMPOSITION COMPRISING FATTY SUBSTANCE, AND USES THEREOF

(71) Applicants: Anne Bouchara, Paris (FR); Gaëlle Petit, Paris (FR)

(72) Inventors: Anne Bouchara, Paris (FR); Gaëlle Petit, Paris (FR)

(21) Appl. No.: 13/642,453

(22) PCT Filed: Apr. 18, 2011
(86) PCT No.: PCT/EP2011/056101

§ 371 Date: Feb. 26, 2013

Related U.S. Application Data
(60) Provisional application No. 61/329,785, filed on Apr. 30, 2010.

(30) Foreign Application Priority Data
Apr. 19, 2010 (FR) 1052953

(51) Int. Cl. A61K 8/19; A61Q 5/04

(52) U.S. Cl. CPC A61K 8/19; A61Q 5/04
USPC 424/70.5; 424/70.2

(57) ABSTRACT

A cosmetic composition for relaxing keratin fibres, comprising an alkaline agent, a fatty phase made up of fatty substance(s), the amount of fatty substance being greater than or equal to 52% by weight, relative to the total weight of said composition, and at least one surfactant in an amount of greater than or equal to 5% by weight, relative to the total weight of said composition. The application also relates to a method for relaxing keratin fibres using this composition, and also to the use of this composition for relaxing keratin fibres.

The present application relates to the field of the alkaline relaxing of keratin fibres.

Many individuals with very curly or frizzy hair wish to have straight hair. One method which makes it possible to relax these types of hair consists of alkaline hair relaxing.

Alkaline hair relaxing consists of hydrolysis of the keratin of the hair with strong alkaline agents, such as inorganic hydroxides, for instance sodium hydroxide, or organic hydroxides, such as guanidine hydroxide. The compositions used generally have a pH ranging from approximately 12 to 14. Consequently, the treatment carried out by means of such compositions often results in damage to the keratin fibre and also often causes discomfort on the scalp during the application of these compositions.

The use of alkaline hair relaxing compositions is known in the prior art, in particular in U.S. Pat. No. 4,237,910. That patent describes alkaline hair relaxing compositions comprising from 3% to 50% of fatty substance. However, the results obtained using these compositions are not totally satisfactory.

The problem addressed in the present invention is that of the obtaining of alkaline hair relaxing compositions which allow relaxing that is at least as good, or even improved, compared with the relaxing obtained with the prior art compositions and which make it possible to preserve keratin fibre integrity; or else that of obtaining alkaline hair relaxing compositions which make it possible to obtain hair relaxing that is equivalent to the hair relaxing obtained with the prior art compositions and which comprise a lower amount of alkaline agents than the amounts of alkaline agents contained in the prior art compositions.

Surprisingly and advantageously, the applicant has demonstrated that the composition according to the present application, which comprises an amount of fatty substance of greater than or equal to 45% and one or more oxyalkylenated surfactants in an amount of greater than or equal to 5% by weight, relative to the total weight of the composition, makes it possible to solve these problems.

A first subject of the present application is a cosmetic composition for relaxing keratin fibres, in particular human keratin fibres such as the hair, comprising one or more alkaline agents of hydroxide type, said composition comprising a fatty phase made up of one or more fatty substances, the amount of fatty substance being greater than or equal to 45% by weight, relative to the total weight of said composition, and one or more oxyalkylenated surfactants in a total amount of greater than or equal to 5% by weight, relative to the total weight of said composition.

A second subject of the present application is a method for relaxing keratin fibres, in particular human keratin fibres such as the hair, in which:
the composition is applied to said keratin fibres,
it is left to stand on the keratin fibres for a period ranging from 5 to 60, and preferably from 5 to 30, minutes,

said keratin fibres are rinsed and, optionally, dried.

A third subject of the present application is the use of the composition according to the present application for relaxing keratin fibres, in particular human keratin fibres such as the hair.

The composition according to the present application, formulated with a level of alkaline agents approximately equal to the levels generally used in the alkaline hair relaxing compositions of the prior art, makes it possible to obtain satisfactory, generally improved, hair straightening effectiveness compared with said prior art compositions, and also makes it possible to obtain better preservation of the integrity of the keratin fibre treated.

For the purpose of the present application, the expression “preservation of the integrity of the fibre” is intended to mean that the intrinsic properties of the hair after relaxing are barely modified. In particular, it is intended to mean that the mechanical properties of tensile strength are improved and/or that the porosity is decreased compared with conventional relaxing.

This can reflect a better preservation of the chemical and/or physical constitution of the fibres.

The composition according to the present application, formulated with a level of alkaline agents which is less than the levels of alkaline agents used in the compositions according to the prior art, makes it possible to obtain relaxing effectiveness equivalent to said prior art compositions.

In addition, the compositions according to the present application are stable over time; they can be stored for several months without modification of their appearance.

Other characteristics, aspects, subjects and advantages of the present invention will emerge more clearly on reading the description and the examples which follow.

The compositions according to the present application comprise a fatty phase made up of fatty substance, the amount of fatty substance being greater than or equal to 45%. The composition according to the present application also generally comprises water, preferably in an amount ranging from 15% to 48% by weight, relative to the total weight of said composition. The composition according to the present invention is generally an emulsion, and preferably an oil-in-water emulsion.

The alkaline agent that can be used in the compositions according to the present invention is chosen from inorganic hydroxides and organic hydroxides.

The inorganic hydroxides can be chosen from alkali metal or alkaline-earth metal hydroxides and transition metal hydroxides. As inorganic hydroxides, mention may, for example, be made of sodium hydroxide, lithium hydroxide, calcium hydroxide, magnesium hydroxide, barium hydroxide, strontium hydroxide, manganese hydroxide and zinc hydroxide.

Among organic hydroxides, mention may be made of guanidinium hydroxide (or guanidine hydroxide).

Among inorganic hydroxides, sodium hydroxide is preferred.

The preferred hydroxide compounds are sodium hydroxide, calcium hydroxide, lithium hydroxide and guanidinium hydroxide.

Among organic hydroxides, use will preferably be made of guanidine hydroxide. Guanidine hydroxide is generally obtained by mixing, at the time of use, guanidine carbonate and calcium hydroxide. According to a first variant, the composition according to the invention contains guanidine carbonate, and then, at the time of use, this composition is mixed with a composition comprising calcium hydroxide, called “activator”.

According to a second variant, the composition according to the present invention contains calcium hydroxide, and then, at the time of use, it is mixed with a composition comprising guanidine carbonate, called “activator”.

The amount of hydroxide-type agents in the composition of the invention is preferably such that said composition has a pH ranging from approximately 12 to 14.

Preferably, the hydroxide-type agent composition ranges from 1% to 10% by weight, relative to the total weight of the composition.

Preferably, the inorganic hydroxides are alkali metal or alkaline-earth metal hydroxides.

The alkali metal or alkaline-earth metal hydroxide concentration ranges from 1% to 2.5%, preferably from 1.5% to 2.2% by weight, relative to the total weight of said composition.

The composition obtained after mixing the guanidine carbonate and the calcium hydroxide comprises an amount of guanidine hydroxide preferably ranging from 2.5% to 6%, better still from 3% to 5% by weight, relative to the total weight of the composition obtained.

The activator can also contain a fatty phase composed of one or more fatty substances chosen from the fatty substances mentioned above.

Fatty Substance

The composition comprises a fatty phase made up of one or more fatty substances.

The term “fatty substance” is intended to mean an organic compound which is insoluble in water at ordinary temperature (25° C.) and at atmospheric pressure (760 mmHg), i.e. a solubility of less than 5%, and preferably less than 1%, even more preferably less than 0.1%. The fatty substances have, in their structure, at least one sequence of at least two siloxane groups or a hydrocarbon-based chain containing at least 6 carbon atoms. In addition, the fatty substances are generally soluble in organic solvents under the same temperature and pressure conditions, for instance chloroform, ethanol, benzene, liquid petroleum jelly or decamethylcyclopentasiloxane.

The fatty substances of the invention are non-etherified with oxyalkylenated or glycerolated groups.

Preferably, they do not contain any carboxylic acid function COOH in their structure.

Preferably, the fatty substances of the invention are chosen from hydrocarbons, fatty alcohols, fatty esters, silicones and fatty ethers, and mixtures thereof.

The fatty substances of the invention may be liquid or non-liquid at ordinary temperature (25° C.) and at atmospheric pressure (760 mmHg; i.e. 1.013×10⁵ Pa).

The liquid fatty substances of the invention preferably have a viscosity of less than or equal to 2 Pa·s, better still less than or equal to 1 Pa·s, and even better still less than or equal to 0.1 Pa·s, at a temperature of 25° C. and at a shear rate of 1 s⁻¹.

The term “liquid hydrocarbon” is intended to mean a hydrocarbon composed solely of carbon and hydrogen atoms, which is liquid at ordinary temperature (25° C.) and at atmospheric pressure (760 mmHg; i.e. 1.013×10⁵ Pa).

More particularly, the liquid hydrocarbons are chosen from:
linear or branched, optionally cyclic, C₆-C₁₆ lower alkanes. By way of example, mention may be made of hexane, undecane, dodecane, tridecane, and isoparaffins, for instance isohexadecane, isododecane and isodecane,
linear or branched hydrocarbons of mineral, animal or synthetic origin, containing more than 16 carbon atoms, such as volatile or non-volatile liquid paraffins, and derivatives thereof, petroleum jelly, liquid petroleum jelly, polydecenes, hydrogenated polyisobutene such as Parleam®, and squalane.

In one preferred variant, the liquid hydrocarbon(s) is (are) chosen from volatile or non-volatile liquid paraffins, and derivatives thereof, and liquid petroleum jelly.

The term “liquid fatty alcohol” is intended to mean a non-glycerolated and non-oxyalkylenated fatty alcohol that is liquid at ordinary temperature (25° C.) and at atmospheric pressure (760 mmHg; i.e. 1.013×10⁵ Pa).

Preferably, the liquid fatty alcohols of the invention comprise from 8 to 30 carbon atoms. The liquid fatty alcohols of the invention may be saturated or unsaturated.

The saturated liquid fatty alcohols are preferably branched. They may optionally comprise in their structure at least one aromatic or non-aromatic ring. They are preferably acyclic.

More particularly, the liquid saturated fatty alcohols of the invention are chosen from octyldodecanol, isostearyl alcohol and 2-hexyldecanol. Octyldodecanol is very particularly preferred.

The liquid unsaturated fatty alcohols have in their structure at least one double or triple bond. Preferably, the fatty alcohols of the invention bear in their structure one or more double bonds. When several double bonds are present, there are preferably 2 or 3 of them, and they may be conjugated or unconjugated.

These unsaturated fatty alcohols may be linear or branched.

They may optionally comprise in their structure at least one aromatic or non-aromatic ring. They are preferably acyclic.

More particularly, the liquid unsaturated fatty alcohols of the invention are chosen from oleyl alcohol, linoleyl alcohol, linolenyl alcohol and undecylenyl alcohol. Oleyl alcohol is very particularly preferred.

The term “liquid fatty ester” is intended to mean an ester derived from a fatty acid and/or from a fatty alcohol and that is liquid at ordinary temperature (25° C.) and at atmospheric pressure (760 mmHg; i.e. 1.013×10⁵ Pa).

The esters are preferably liquid esters of saturated or unsaturated, linear or branched C₁-C₂₆ aliphatic mono- or polyacids and of saturated or unsaturated, linear or branched C₁-C₂₆ aliphatic mono- or polyalcohols, the total number of carbon atoms of the esters being greater than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, isopropyl palmitate, alkyl myristates such as isopropyl myristate or ethyl myristate, isocetyl stearate, ethylhexyl isononanoate, isodecyl neopentanoate and isostearyl neopentanoate.

Esters of C₄-C₂₂ dicarboxylic or tricarboxylic acids and of C₁-C₂₂ alcohols and esters of mono-, di- or tricarboxylic acids and of non-sugar C₄-C₂₆ di-, tri-, tetra- or pentahydroxy alcohols may also be used.

Mention may in particular be made of: diethyl sebacate; diisopropyl sebacate; bis(2-ethylhexyl)sebacate; diisopropyl adipate; di-n-propyl adipate; dioctyl adipate; bis(2-ethylhexyl)adipate; diisostearyl adipate; bis(2-ethylhexyl)maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

The composition may also comprise, as a liquid fatty ester, sugar esters and diesters of C₆-C₃₀ and preferably C₁₂-C₂₂ fatty acids. It is recalled that the term “sugar” is intended to mean oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen in particular from the group comprising the esters or mixtures of esters of sugars described previously and of linear or branched C₆-C₃₀ and preferably C₁₂-C₂₂ fatty acids. If they are unsaturated, these compounds may comprise one to three conjugated or unconjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

Finally, natural or synthetic esters of mono-, di- or triacids with glycerol may also be used. Among these, mention may be made of plant oils.

As oils of plant origin or synthetic triglycerides that may be used in the composition of the invention as liquid fatty esters, mention may be made, for example, of triglyceride oils of plant or synthetic origin, such as liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

Liquid fatty esters derived from monoalcohols will preferably be used as esters according to the invention. Isopropyl myristate or isopropyl palmitate is particularly preferred.

The term “liquid silicone” is intended to mean an organo-polysiloxane which is liquid at ordinary temperature (25° C.) and at atmospheric pressure (760 mmHg; i.e. 1.013×10⁵ Pa).

Preferably, the silicone is chosen from liquid polydialkylsiloxanes, especially liquid polydimethylsiloxanes (PDMS) and liquid polyorganosiloxanes comprising at least one aryl group.

These silicones may also be organomodified. The organomodified silicones that can be used in accordance with the invention are liquid silicones as defined above and comprising in their structure one or more organofunctional groups attached via a hydrocarbon-based group.

Organopolysiloxanes are defined in greater detail in Walter Noll's “Chemistry and Technology of Silicones” (1968) Academic Press. They may be volatile or non-volatile.

When they are volatile, the silicones are more particularly chosen from those having a boiling point of between 60° C. and 260° C., and even more particularly from:

(i) cyclic polydialkylsiloxanes comprising from 3 to 7, preferably from 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof.

Mention may also be made of cyclocopolymers of the dimethylsiloxane/methylalkylsiloxane type, such as Volatile Silicone® FZ 3109 sold by the company Union Carbide, of formula:

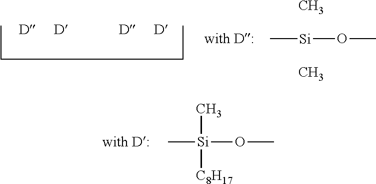

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers “Volatile Silicone Fluids for Cosmetics”. The viscosity of the silicones is measured at 25° C. according to ASTM standard 445 Appendix C.

Non-volatile polydialkylsiloxanes may also be used.

These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name Dimethiconol (CTFA), such as the oils of the 48 series from the company Rhodia.

Among the silicones containing aryl groups are polydiarylsiloxanes, especially polydiphenylsiloxanes, and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil DOW CORNING 556 COSMETIC GRADE FLUID from Dow Corning;
the silicones of the PK series from Bayer, such as the product PK20;
certain oils of the SF series from General Electric, such as SF 1023, SF 1154, SF 1250 and SF 1265.

The organomodified liquid silicones may in particular contain thiol groups.

The liquid fatty ethers are chosen from liquid dialkyl ethers such as dicaprylyl ether.

The fatty substances may be non-liquid at ambient temperature and at atmospheric pressure.

The term “non-liquid” is preferably intended to mean a compound that is solid or a compound that has a viscosity of greater than 2 Pa·s at a temperature of 25° C. and at a shear rate of 1 s⁻¹.

More particularly, the non-liquid fatty substances are chosen from fatty alcohols, esters of fatty acids and/or of fatty alcohols, non-silicone waxes, silicones and fatty ethers, which are non-liquid and preferably solid.

The non-liquid fatty alcohols that are suitable for use in the invention are more particularly chosen from saturated or unsaturated, linear or branched alcohols comprising from 8 to 30 carbon atoms. Mention may be made, for example, of cetyl alcohol, stearyl alcohol and a mixture thereof (cetylstearyl alcohol).

As regards the non-liquid esters of fatty acids and/or of fatty alcohols, mention may in particular be made of solid esters derived from C₉-C₂₆ fatty acids and from C₉-C₂₆ fatty alcohols.

Among these esters, mention may be made of octyldodecyl behenate; isocetyl behenate; cetyl lactate; stearyl octanoate; octyl octanoate; cetyl octanoate; decyl oleate; myristyl stearate; octyl palmitate; octyl pelargonate; octyl stearate; alkyl myristates such as cetyl, myristyl or stearyl myristate; hexyl stearate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may be made especially of: diethyl sebacate; diisopropyl sebacate; diisopropyl adipate; di-n-propyl adipate; dioctyl adipate; dioctyl maleate.

Among all the additional esters mentioned above, it is preferred to use myristyl, cetyl or stearyl palmitates, alkyl myristates such as cetyl myristate, and stearyl myristyl myristate.

The (non-silicone) wax(es) is (are) selected in particular from carnauba wax, candelilla wax, esparto grass wax, paraffin wax, ozokerite, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that may be used according to the invention are especially marine waxes such as the product sold by the company Sophim under the reference M82, and polyethylene waxes or polyolefin waxes in general.

The non-liquid silicones in accordance with the invention may be in the form of waxes, resins or gums.

Preferably, the non-liquid silicone is chosen from polydialkylsiloxanes, especially polydimethylsiloxanes (PDMS), and organomodified polysiloxanes comprising at least one functional group chosen from poly(oxyalkylene) groups, amino groups and alkoxy groups.

The silicone gums that can be used in accordance with the invention are especially polydialkylsiloxanes and preferably polydimethylsiloxanes with high number-average molecular weights of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Products that can be used more particularly in accordance with the invention are mixtures such as:
mixtures formed from a polydimethylsiloxane hydroxylated at the chain end, or dimethiconol (CTFA) and from a cyclic polydimethylsiloxane also known as cyclomethicone (CTFA), such as the product Q2 1401 sold by the company Dow Corning;
mixtures of a polydimethylsiloxane gum and a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs with different viscosities, and more particularly of a PDMS gum and a PDMS oil, such as the product SF 1236 from the company General Electric. The product SF 1236 is a mixture of a gum SE 30 defined above, with a viscosity of 20 m²/s, and of an oil SF 96 with a viscosity of 5×10⁻⁶ m²/s. This product preferably comprises 15% of gum SE 30 and 85% of an oil SF 96.

The organopolysiloxane resins that can be used in accordance with the invention are crosslinked siloxane systems containing the following units:

R₂SiO_{2/2}, R₃SiO_{1/2}, RSiO_{3/2} and SiO_{4/2}

in which R represents an alkyl containing 1 to 16 carbon atoms. Among these products, the ones that are particularly preferred are those in which R denotes a C₁-C₄ lower alkyl group, more particularly methyl.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

Among the organomodified silicones, mention may be made of polyorganosiloxanes comprising:
substituted or unsubstituted amino groups, for instance the products sold under the names Q2 8220 and Dow Corning 929 or 939 by the company Dow Corning. The substituted amino groups are in particular C₁-C₄ aminoalkyl groups;
alkoxylated groups, for instance the product sold under the names Abil Wax® 2428, 2434 and 2440 by the company Goldschmidt.

The non-liquid fatty ethers are chosen from dialkyl ethers and especially dicetyl ether and distearyl ether, alone or as a mixture.

Preferably, the composition according to the invention contains one or more fatty substances that are liquid at ordinary temperature (25° C.) and at atmospheric pressure (760 mmHg; i.e. 1.013×10⁵ Pa), optionally combined with one or more fatty substances that are non-liquid under the same conditions.

Even more preferably, the fatty substance is a plant oil or a liquid hydrocarbon.

Preferably, the fatty phase comprises a mineral oil and even more preferably a liquid petroleum jelly.

The fatty phase advantageously comprises at least one other fatty substance other than a mineral oil, preferably chosen from fatty alcohols.

The fatty alcohols suitable for use in the invention are more particularly chosen from saturated or unsaturated, linear or branched alcohols comprising from 8 to 30 carbon atoms; mention may, for example, be made of cetyl alcohol, stearyl alcohol and a mixture thereof (cetylstearyl alcohol), octyldodecanol, 2-butyloctanol, 2-hexyldecanol, 2-undecylpentadecanol, oleyl alcohol and linoleyl alcohol. The fatty alcohol used is preferably cetylstearyl alcohol.

Preferably, the fatty phase is a mixture of a liquid petroleum jelly and of a fatty alcohol, preferably cetylstearyl alcohol.

The composition according to the invention comprises a fatty phase in a content preferably greater than or equal to 52% by weight, the amount of fatty phase preferably ranging from 52% to 70%, and even more preferably from 55% to 70% by weight, relative to the total weight of the composition.

Surfactants

The composition according to the present invention also comprises one or more oxyalkylenated surfactants. These surfactants can be chosen from cationic surfactants, anionic surfactants, nonionic surfactants and amphoteric surfactants.

Preferably, the surfactant(s) is (are) chosen from nonionic or anionic surfactants.

The term “anionic surfactant” is intended to mean a surfactant comprising, as ionic or ionizable groups, only anionic groups. These anionic groups are preferably chosen from the following groups: CO₂H, CO₂⁻, SO₃H, SO₃⁻, OSO₃H, OSO₃⁻, H₂PO₃, HPO₃⁻, PO₃²⁻, H₂PO₂, HPO₂, HPO₂⁻, PO₂⁻, POH and PO⁻.

By way of examples of anionic surfactants that can be used in the composition according to the invention, mention may be made of alkyl sulphates, alkyl ether sulphates, alkylamido ether sulphates, alkylarylpolyether sulphates, monoglyceride sulphates, alkylsulphonates, alkylamidesulphonates, alkylarylsulphonates, alpha-olefin sulphonates, paraffin sulphonates, alkylsulphosuccinates, alkyl ether sulphosuccinates, alkylamide sulphosuccinates, alkylsulpho-acetates, acylsarcosinates, acylglutamates, alkylsulphosuccinamates, acylisethionates and N-acyltaurates, salts of alkyl monoesters and of polyglycoside-polycarboxylic acids, acyllactylates, D-galactosiduronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds comprising from 6 to 24 carbon atoms and the aryl group denoting a phenyl group.

These compounds may be oxyethylenated and then preferably comprise from 1 to 50 ethylene oxide units.

The salts of C₆-C₂₄ alkyl monoesters of polyglycoside-polycarboxylic acids can be chosen from C₆-C₂₄ alkyl polyglycoside-citrates, C₆-C₂₄ alkyl polyglycoside-tartrates and C₆-C₂₄ alkyl polyglycoside-sulphosuccinates.

When the anionic surfactant(s) is (are) in salt form, it (they) may be chosen from alkali metal salts such as the sodium or potassium salt and preferably the sodium salt, the ammonium salts, the amine salts and in particular amino alcohol salts or the alkaline-earth metal salts such as the magnesium salt.

By way of examples of amino alcohols, mention may in particular be made of mono-, di- and triethanolamine salts, mono-, di- or triisopropanolamine salts, 2-amino-2-methyl-1-propanol salts, 2-amino-2-methyl-1,3-propanediol salts and tris(hydroxymethyl)amino-methane salts.

Alkali metal or alkaline-earth metal salts, and in particular sodium or magnesium salts, are preferably used.

The anionic surfactants are preferably chosen from sodium alkyl sulphates.

The nonionic surfactants are more particularly chosen from monooxyalkylenated or polyoxyalkylenated, or polyglycerolated nonionic surfactants. The oxyalkylene units are more particularly oxyethylene or oxypropylene units, or a combination thereof, preferably oxyethylene units.

Examples of oxyalkylenated nonionic surfactants that may be mentioned include:
oxyalkylenated (C₈-C₂₄)alkylphenols,
saturated or unsaturated, linear or branched, oxyalkylenated C₈-C₃₀ alcohols, and in particular ethers of polyethylene glycol and of C₈-C₃₀ fatty alcohols,
saturated or unsaturated, linear or branched, oxyalkylenated C₈-C₃₀ amides,
esters of saturated or unsaturated, linear or branched C₈-C₃₀ acids and of polyethylene glycols,
polyoxyethylenated esters of saturated or unsaturated, linear or branched C₈-C₃₀ acids and of sorbitol,
saturated or unsaturated, oxyethylenated plant oils,
condensates of ethylene oxide and/or of propylene oxide, inter alia,
alkylpolyglycosides,
N-alkylglucamine derivatives,
oxyethylenated and/or oxypropylenated polydimethyl-siloxanes,
or mixtures thereof.

The surfactants have a number of moles of ethylene oxide and/or of propylene oxide of between 1 and 100, preferably between 2 and 50. Advantageously, the nonionic surfactants do not comprise any oxypropylene units.

In accordance with one preferred embodiment of the invention, the nonionic surfactants are chosen from ethers of polyethylene glycol and of a fatty alcohol preferably comprising from 2 to 35, preferably from 2 to 30 and even more preferably 10 oxyethylene groups and the fatty chain of which preferably comprises from 10 to 25, preferably from 16 to 22 and even more preferably 22 carbon atoms.

Advantageously, the nonionic surfactants are chosen from steareth-2, ceteth-10 and beheneth, in particular beheneth-5, beheneth- and beheneth-30, beheneth-10 being the preferred nonionic surfactant.

Advantageously, the composition according to the invention comprises a single surfactant.

The composition according to the invention comprises an amount of surfactant(s) of greater than or equal to 5% by weight; preferably, the amount of surfactant(s) ranges from 5% to 20% and even more preferably from 10% to 15% by weight, relative to the total weight of the composition.

The composition according to the present invention also preferably comprises one or more additional agents chosen from:
conditioning agents other than the fatty substances of the invention, such as cationic polymers or cationic surfactants,
thickening polymers of natural or synthetic origin,
swelling agents and penetrating agents,
hydrophobic or hydrophilic solvents,
direct dyes, in particular cationic direct dyes or natural direct dyes,
oxidation dyes,
organic pigments or mineral pigments,
UV screening agents,
fillers such as, for example, pearlescent agents, TiO₂, resins or clays,
fragrances.

Another subject of the present application is a method for relaxing keratin fibres, in particular human keratin fibres such as the hair, in which:
the composition is applied to said keratin fibres,
it is left to stand on the hair for a period ranging from 5 to 60, and preferably from 5 to 30 minutes,

said keratin fibres are rinsed and, optionally, dried.

The step of applying the composition comprising one or more alkaline agents of hydroxide type can be carried out under heat and in particular at a temperature ranging from 40 to 250° C. by means of any heating apparatus, such as hairdryers, hoods or irons.

The application of the composition comprising one or more alkaline agents of hydroxide type can be followed by the application of an oxidizing composition comprising one or more oxidizing agents, and in particular hydrogen peroxide.

Finally, another subject of the present application is the use of the composition according to the present application for relaxing keratin fibres, in particular human keratin fibres such as the hair.

The following examples are given by way of illustration of the present invention, and cannot limit the scope thereof.

### EXAMPLES

### 1—Examples of Hair Relaxing Compositions Based on Sodium Hydroxide

### 1.1. Examples 1 and 2

### Composition Based on Beheneth-10

a—Composition and Preparation Method:

The hair relaxing compositions according to Examples 1 and 2 (see Table 1) are prepared in the following way:

Part A of Example 1 comprises 28 g of water and that of Example 2 comprises 30 g of water.

In order to prepare part B of Examples 1 and 2, the cetearyl alcohol and the beheneth-10 are melted at 70° C. in the presence of liquid petroleum jelly.

Finally, for part C of Examples 1 and 2, a 30% sodium hydroxide solution is prepared by dissolving sodium hydroxide pellets in water.

Parts A and B are heated at 70° C. with stirring for 10 minutes. The mixture is then cooled to 40° C. and part C is then added with stirring to the previous mixture.

TABLE 1
Composition of Examples 1 and 2

| | Composition | Example 1 | Example 2 |
|---|---|---|---|
| PART A | Distilled water | 28 | 30 |
| PART B | Liquid | 50 | 10 |
| | petroleum jelly | | |
| | Cetearyl | 5 | 50 |
| | alcohol | | |
| | Beheneth-10 | 10 | 5 |
| PART C | Distilled water | 5 | 3.5 |
| | Sodium | 2 | 1.5 |
| | hydroxide | | |

The liquid petroleum jelly is supplied by Exonnmobil and has the commercial reference Narcol 82.

The cetearyl alcohol is supplied by Cognis and has the commercial reference Lanette O OR.

The beheneth-10 is supplied by the company Cognis under the commercial reference Eumulgin BA 10.

b—Evaluation on Heads:

Evaluation of the Composition According to Example 1:

The composition according to Example 1, which contains 55% of fatty phase, is evaluated on 5 models different from those of Example 1, compared with a Dark and Lovely Precise Essential Relaxer Regular formula, the fatty phase of which represents 36% of the formula. It should be noted that these two formulas contain comparable amounts of sodium hydroxide (respectively 2% for the invention and 2.1% for the comparative product).

The products are applied to models in a half-head test, one of the compositions being applied on the right and the other on the left. The amount of product applied on each side is approximately 130 g. The leave-in time is 15 minutes.

A panel of experts evaluates the relaxing effectiveness on dry hair and assigns a mark of between 0 and 50; 0 corresponding to an absence of hair relaxing and 50 to very good hair relaxing.

Average Relaxing Marks on Dry Hair:

Dark and Lovely Precise Essential Relaxer Regular: 38

Composition according to Example 1: 46

The hair relaxing effectiveness of the composition according to Example 1 is therefore greater than that of the prior art composition, at similar sodium hydroxide levels.

Evaluation of the Composition According to Example 2:

The composition according to Example 2, which contains 55% of fatty phase, is evaluated on 5 models different from those of Example 1, compared with a Dark and Lovely Precise Essential Relaxer Regular formula, the fatty phase of which represents 36% of the formula. The composition according to Example 1 contains an amount of alkaline active agent which is significantly less than the Dark and Lovely Precise Essential Relaxer Regular formula (1.5% compared with 2.1%).

The products are applied to models in a half-head test, one of the compositions being applied on the right and the other on the left. The amount of product applied on each side is approximately 130 g. The leave-in time is 15 minutes. A panel of experts evaluates the relaxing effectiveness on dry hair and assigns a mark of between 0 and 50; 0 corresponding to an absence of hair relaxing and 50 to very good hair relaxing.

Average Relaxing Marks on Dry Hair:

Dark and Lovely Precise Essential Relaxer Regular: 38

Composition according to Example 2: 41

The hair relaxing effectiveness of the composition according to Example 2 is therefore equivalent to that of the prior art composition, at a lower sodium hydroxide level.

### 1.2. Examples 4 and 5

### Compositions Based on Guanidine Carbonate

a—Compositions and Preparation Method:

The hair relaxing composition according to Example 4 is prepared in the following way: the guanidine carbonate is mixed with water, with stirring, at 70° C.; part A.

In another part, the cetearyl alcohol and the beheneth-10 are melted at 70° C. in the presence of liquid petroleum jelly: part B.

Parts A and B are heated at 70° C. with stirring for 10 minutes. The mixture is then cooled with stirring.

Parts A and B of the hair relaxing composition according to Example 5 are prepared in the same way as those of Example 4, and then part C is subsequently added, cold, with stirring, to the previous mixture.

Base Cream

TABLE 2
Compositions of Examples 4 and 5

| | Composition | Example 4 | Example 5 |
|---|---|---|---|
| PART A | Distilled water | 28.6 | 26.6 |
| | Guanidine | 6.4 | 6.4 |
| | carbonate | | |
| PART B | Liquid | 50 | 50 |
| | petroleum jelly | | |
| | Cetearyl | 5 | 5 |
| | alcohol | | |
| | Beheneth-10 | 10 | 10 |
| PART C | PEG-14 | 0 | 2 |
| | dimethicone | | |

The liquid petroleum jelly is supplied by Exonnmobil and has the commercial reference Narcol 82.

The cetearyl alcohol is supplied by Cognis and has the commercial reference Lanette O OR.

The beheneth-10 is supplied by the company Cognis under the commercial reference Eumulgin BA 10.

The PEG-14 dimethicone is supplied by the company Evonik under the commercial reference Abil B 8842.

Activator

TABLE 3
Composition of the activator

| Composition | Activator |
|---|---|
| Fumed silica | 3 |
| Propylene glycol | 52 |
| Calcium hydroxide | 45 |

b—Evaluation on Heads:

Evaluation of the Composition According to Example 4:

92.6 g of base cream according to Example 4, which contains 55% of fatty phase, are mixed with 7.4 g of activator. The obtained composition contains 50.9% of fatty substances and 5% by weight of hydroxide carbonate.

In addition, 92.6 g of base cream of Dark and Lovely Regular No-Lye Conditioning Relaxer System, which contains 30% of fatty phase, and 7.4 g of activator of Dark and Lovely Regular are mixed.

After mixing, the Dark and Lovely Regular No-Lye Conditioning Relaxer System contains 5% by weight of guanidine hydroxide.

It should be noted that these two formulas contain the same amount of guanidine carbonate and of calcium hydroxide.

The two formulas are compared on 13 models.

The products are applied to models in a half-head test, one of the compositions being applied on the right and the other on the left. The amount of product applied on each side is approximately 130 g. The leave-in time is 20 minutes.

A panel of experts evaluates the relaxing effectiveness on dry hair and assigns a mark of between 0 and 50; 0 corresponding to an absence of hair relaxing and 50 to very good hair relaxing.

Average Relaxing Marks on Dry Hair:

Dark and Lovely Regular No-Lye Conditioning Relaxer System: 33

Composition according to Example 4: 40

The hair relaxing effectiveness of the composition according to Example 4 is therefore greater than that of the prior art composition, at the same level of guanidine derivative.

Evaluation of the Composition According to Example 5:

92.6 g of base cream according to Example 5, which contains 55% of fatty phase, are mixed with 7.4 g of activator. The obtained composition contains 50.9% by weight of fatty substances and 5% by weight of hydroxide carbonate.

In addition, 92.6 g of base cream of Dark and Lovely Regular No-Lye Conditioning Relaxer System activator, which contains 30% of fatty phase, and 7.4 g of activator of Dark and Lovely Regular are mixed.

After mixing, the Dark and Lovely Regular No-Lye Conditioning Relaxer System contains 5% by weight of guanidine hydroxide.

It should be noted that these two formulas contain the same amount of guanidine carbonate and of calcium hydroxide.

The two formulas are compared on 9 models different from those of Example 4.

The products are applied to models in a half-head test, one of the compositions being applied on the right and the other on the left. The amount of product applied on each side is approximately 130 g. The leave-in time is 20 minutes. A panel of experts evaluates the relaxing effectiveness on dry hair and assigns a mark of between 0 and 50; 0 corresponding to an absence of hair relaxing and 50 to very good hair relaxing.

Average Relaxing Marks on Dry Hair:

Dark and Lovely Regular No-Lye Conditioning Relaxer System: 35

Composition according to Example 5: 43

The hair relaxing effectiveness of the composition according to Example 5 is therefore greater than that of the prior art composition, at the same level of guanidine derivative.

### 1.3. Examples 6 and 7

### Compositions Based on Guanidine Carbonate

a—Compositions and Preparation Method:

The hair relaxing compositions according to Example 6 and 7 are prepared in the following way: the guanidine carbonate is mixed with water, with stirring, at 70° C.; part A.

In another part, the cetearyl alcohol and the beheneth-10 are melted at 70° C. in the presence of liquid petroleum jelly: part B.

Parts A and B are heated at 70° C. with stirring for 10 minutes. The mixture is then cooled with stirring and then part C is subsequently added, cold, with stirring, to the previous mixture.

TABLE 4
Compositions of Examples 6 and 7

| | Composition | Example 6 | Example 7 |
|---|---|---|---|
| PART A | Distilled water | 27.3 | 27.9 |
| | Guanidine | 5.7 | 5.1 |
| | carbonate | | |
| PART B | Liquid | 50 | 50 |
| | petroleum jelly | | |
| | Cetearyl | 5 | 5 |
| | alcohol | | |
| | Beheneth-10 | 10 | 10 |
| PART C | PEG-14 | 2 | 2 |
| | dimethicone | | |

The liquid petroleum jelly is supplied by Exonnmobil and has the commercial reference Narcol 82.

The cetearyl alcohol is supplied by Cognis and has the commercial reference Lanette O OR.

The beheneth-10 is supplied by the company Cognis under the commercial reference Eumulgin BA 10.

The PEG-14 dimethicone is supplied by the company Evonik under the commercial reference Abil B 8842.

Activator

TABLE 5
Composition of the activator

| Composition | Activator |
|---|---|
| Fumed silica | 3 |
| Propylene glycol | 52 |
| Calcium hydroxide | 45 |

b—Evaluation on Heads:

Evaluation of the Composition According to Example 6:

92.6 g of base cream according to Example 6, which contains 55% of fatty phase, are mixed with 7.4 g of activator. The obtained composition contains 50.9% of fatty substances and 4.45% by weight of guanidine hydroxide.

In addition, 92.6 g of base cream of Dark and Lovely Regular No-Lye Conditioning Relaxer System, which contains 30% of fatty phase, and 7.4 g of activator of Dark and Lovely Regular are mixed.

After mixing, the Dark and Lovely Regular No-Lye Conditioning Relaxer System contains 5% by weight of guanidine hydroxide and 27.8% by weight of fatty substances.

It should be noted that the formula according to the invention contains 5.7% by weight of guanidine carbonate and the commercial reference contains 6.4% by weight of guanidine carbonate.

The two formulas are compared on 10 models.

The products are applied to models in a half-head test, one of the compositions being applied on the right and the other on the left. The amount of product applied on each side is approximately 130 g. The leave-in time is 20 minutes.

A panel of experts evaluates the relaxing effectiveness on dry hair and assigns a mark of between 0 and 10; 1 to very good hair relaxing and 10 corresponding to an absence of hair relaxing.

Average Relaxing Marks on Dry Hair:

Dark and Lovely Regular No-Lye Conditioning Relaxer System: 2

Composition according to Example 6: 1.7

The hair relaxing effectiveness of the composition according to Example 6 is at least as good as that of the prior art composition, at a lower guanidine derivative level.

The low amount of guanidine derivative used in the composition according to Example 6 allows the preservation of the integrity of the fibre.

Indeed, mechanical tests of tensile strength show that the Young's modulus of relaxed hair by the composition according to example 6 is higher (0.801±0.154 GPa) than that of the relaxed hair by the commercial reference (0.53±0.101 GPa).

Evaluation of the Composition According to Example 7:

88 g of base cream according to Example 7, which contains 55% of fatty phase, are mixed with 12 g of activator. The obtained composition contains 48.4% of fatty substances and 3.8% by weight of guanidine hydroxide.

In addition, 88 g of base cream of Dark and Lovely No Lye Classic Base Relaxer Color Treated, which contains 30% by weight of fatty phase, and 12 g of activator of Dark and Lovely Regular are mixed. After mixing, the obtained composition contains 26.5% by weight of fatty substances.

It should be noted that the formula according to the invention contains 5.1% by weight of guanidine carbonate and the commercial reference contains 5.7% by weight of guanidine carbonate.

The two formulas are compared on 8 models.

The products are applied to models in a half-head test, one of the compositions being applied on the right and the other on the left. The amount of product applied on each side is approximately 130 g. The leave-in time is 20 minutes.

A panel of experts evaluates the relaxing effectiveness on dry hair and assigns a mark of between 1 and 10; 1 corresponding to very good hair relaxing and 10 corresponding to frizzy hair.

Average Relaxing Marks on Dry Hair:

Dark and Lovely No Lye Classic Base Relaxer Color Treated: 2.4

Composition according to Example 7: 1.9

The hair relaxing effectiveness of the composition according to Example 7 is therefore greater than that of the prior art composition, at a lower level of guanidine derivative.

1. Cosmetic composition for relaxing keratin fibres, comprising one or more alkaline agents of hydroxide type, characterized in that it comprises a fatty phase made up of one or more fatty substances, the amount of fatty substance being greater than or equal to 45% by weight, relative to the total weight of said composition, and one or more oxyalkylenated surfactants in an amount of greater than or equal to 5% by weight, relative to the total weight of said composition.

2. Cosmetic composition according to claim 1, in which the alkaline agent of hydroxide type is chosen from inorganic hydroxides, preferably from alkali metal or alkaline-earth metal hydroxides and transition metal hydroxides, and in particular sodium hydroxide.

3. Cosmetic composition according to claim 1, in which the alkaline agent of hydroxide type is an organic hydroxide, and preferably guanidine hydroxide.

4. Composition according to claim 1 any one of the preceding claims, in which the fatty phase comprises one or more fatty substances chosen from hydrocarbons, fatty alcohols, fatty esters, silicones and fatty ethers, and mixtures thereof, which are preferably liquid.

5. Composition according to claim 1, in which the fatty phase comprises a mineral oil, and even more preferably a liquid petroleum jelly.

6. Composition according to claim 5, comprising at least one other fatty substance other than a mineral oil, chosen from fatty alcohols and preferably cetylstearyl alcohol.

7. Composition according to claim 1, in which the amount of fatty phase is greater than or equal to 52%, ranges from 52% to 70%, and even more preferably from 55% to 70% by weight, relative to the total weight of the composition.

8. Composition according to claim 1, characterized in that the surfactant is an anionic or nonionic surfactant.

9. Composition according to claim 8, characterized in that the surfactant is an anionic surfactant chosen from sodium alkyl sulphates.

10. Composition according to claim 8, characterized in that the surfactant is a nonionic surfactant chosen from ethers of polyethylene glycol and of a fatty alcohol preferably comprising from 2 to 35, preferably from 2 to 30 and even more preferably 10 oxyethylene groups and the fatty chain of which preferably comprises from 10 to 25, preferably from 16 to 22 and even more preferably 22 carbon atoms.

11. Composition according to claim 1, characterized in that it comprises a single surfactant.

12. Composition according to claim 1, characterized in that the amount of surfactant(s) ranges from 5% to 20% and even more preferably from 10% to 15% by weight, relative to the total weight of the composition.

13. Composition according to claim 1, characterized in that it comprises one or more additional agents chosen from:
conditioning agents other than the fatty substances,
thickening polymers of natural or synthetic origin, preferably chosen from cationic polymers and cationic surfactants,
swelling agents and penetrating agents,
hydrophobic or hydrophilic solvents,
direct dyes,
oxidation dyes,
organic pigments or mineral pigments,
UV screening agents,
fillers,
fragrances.

14. Method for relaxing keratin fibres, in which:
the composition according to claim 1 is applied to said keratin fibres,
it is left to stand on the keratin fibres for a period ranging from 5 to 20 minutes,
said keratin fibres are optionally rinsed and/or dried.

15. Use of a composition according to claim 1, for relaxing keratin fibres.
